(19) United States
(12) Patent Application Publication
(10) Pub. No.: US 2025/0200342 A1
(43) Pub. Date: Jun. 19, 2025

(54) SYNTHETIC TIME-SERIES DATA GENERATION AND ITS USE IN SURVIVAL ANALYSIS AND SELECTION OF DRUG FOR FURTHER DEVELOPMENT

(71) Applicant: Hoffmann-La Roche Inc., Little Falls, NJ (US)

(72) Inventors: Marta BATLLE LÓPEZ, Basel (CH); Gregory James CHURCH, Basel (CH); Lawrence Anthony DENISON-HALL, Welwyn Garden City (GB); Danilo GUERRERA, Basel (CH); Finn JANSON, Welwyn Garden City (GB)

(21) Appl. No.: 19/069,123

(22) Filed: Mar. 3, 2025

Related U.S. Application Data
(63) Continuation of application No. PCT/EP2023/074333, filed on Sep. 5, 2023.

(30) Foreign Application Priority Data
Sep. 5, 2022 (EP) 22193969.7

(51) Int. Cl. G06N 3/0475; G06N 3/0455; G06N 3/0895; G06N 3/094

(52) U.S. Cl. CPC G06N 3/0475; G06N 3/0455; G06N 3/0895; G06N 3/094

(57) ABSTRACT

A computer-implemented method is provided for generating a trained TTS-GAN which may be used to generate synthetic longitudinal data for use in survival analysis, a clinical trial or clinical research. The TTS-GAN is configured to generate synthetic time-series data based on synthetic context data generated using a machine-learning model by virtue of being trained using training data comprising real context data and added noise data. A technique for executing survival analysis is also provided, which relies on the synthetic longitudinal data.

### TECHNICAL FIELD OF THE INVENTION

The present invention relates to computer-implemented methods of generating a TTS-GAN which may be used to generate synthetic longitudinal data based on input context data. The invention also relates to the generation of synthetic longitudinal data using the TTS-GAN, and the use of the synthetic longitudinal data in survival analysis. Analogous computer programs, computer-readable media, and modules/systems are also provided.

### BACKGROUND TO THE INVENTION

Many technological processes now rely on large amounts of data. For example, large amounts of data are often required to train machine learning models, in order to generate useful results from “real-life” data. Often, large amounts of data are required to, for example, reliable train a machine-learning model, and such large amounts of data, particularly in the context of e.g. rare diseases or other conditions, may not be available. It is therefore sometimes necessary to generate, based on real-life data, to generate synthetic data which has the same, or similar, statistical properties, as the real-life data. Techniques for generation of time-independent, tabular data are known.

Survival analysis is a branch of statistics which is used to analyse and/or estimate the amount of time until an event happens. In a clinical context, this can often refer to a prediction of the death of a biological organism, or a time at which some threshold condition (e.g. onset of a disease or symptom, or organ failure) is met. In engineering contexts, survival analysis is often referred to as “reliability analysis”, and can be used to predict e.g. the failure of a mechanical or electrical system, e.g. based on a threshold condition based on a measurable parameter of the system. Such analysis is clearly highly-dependent on time-series data. At present, there is no way of reliably generating high-quality synthetic time-series data. The present invention was devised in light of this gap.

### SUMMARY OF THE INVENTION

At a high level, the present invention is directed towards a computer-implemented method of generating synthetic time-series data or synthetic longitudinal data, which relies on a modified version of a transformer-based time series generative adversarial network (referred to herein as a “TTS-GAN”), such as the one described in detail in Li et al. (2022)¹. Unlike in Li et al. (2022), however, the TTS-GAN employed in the present invention is trained to establish a relationship between non-time-series data (referred to herein as “context data”) and the time-series data. The present invention covers both the generation of the modified TTS-GAN, and separately, its use in generating high-quality synthetic longitudinal data. ¹https://doi.org/10.48550/arXiv.2202.02691

A first aspect of the present invention provides a computer-implemented method of generating a TTS-GAN configured to generate synthetic longitudinal data for use in survival analysis, or for use in a clinical trial, or for use in clinical research, the TTS-GAN comprising a TTS-GAN generator and a TTS-GAN discriminator. The computer-implemented method comprises: receiving longitudinal training data comprising context data and time-series data; training the TTS-GAN using the longitudinal training data, wherein training the TTS-GAN comprises: (a) training the TTS-GAN generator to generate synthetic time-series data using the context data and added noise; (b) training the TTS-GAN discriminator to discriminate between the time-series data and the synthetic time-series data generated by the TTS-GAN generator; (c) repeating steps (a) and (b) alternately until a predetermined end condition is met. From this, it will be appreciated that by training the TTS-GAN generator to generate synthetic time-series data using only context data and added noise, the TTS-GAN generator is able to learn the relationship between the context data and the time-series data. In step (a), the TTS-GAN may generate a plurality of time-series for each piece of input context training data and added noise. The computer-implemented method may further comprise a step of outputting and/or storing the trained TTS-GAN after step (c). In step (c), steps (a) and (b) may be repeated a predetermined number of times (i.e. the training may progress for a predetermined number of epochs). The number of epochs may be no less than 1000, no less than 2000, no less than 3000, no less than 4000, or no less than 5000, for example. Alternatively, training may stop when a predetermined convergence criterion is met.

Instead of determining whether a GAN has converged, instead, it is possible to analyse the loss of both the generator and discriminator to determine whether the model is not converging. This concept may then be exploited to terminate training early when (i.e. a few epochs before the discriminator and generator loss stopped converging).

The two main instances of nonconvergence would be mode collapse or vanishing gradient:
Mode collapse occurs when the generator becomes too good (loss is low) vs the discriminator performance (loss is high) whereby the generator will simply learn to generate low-diversity and high fidelity data (e.g., one single sample) that fools the discriminator every time. This is not ideal as we want our synthetic data to represent the original population.
Vanishing gradient is the opposite problem whereby the discriminator becomes too good at predicting real and fake to the extent that the generator cannot improve (low discriminator loss, high generator loss).

During training, if neither of these instances of nonconvergence occur, then it is safe to continue training the model. Otherwise, training should be terminated and the extracted model should be from the last epoch where neither mode collapse nor vanishing gradient appeared to occur.

In the present application, “context data” is used to refer to non-time-series data, preferably but not necessarily numerical (or similar) data describing an attribute of a particular individual or subject. Thus, context data may refer to data describing an attribute which remains constant, or approximately constant with time. Examples of context data might include date-of-birth, height, weight (unless variation of weight with time is being considered, in which case, weight might be considered as part of time-series data). Context data may include binary data to indicate e.g. the presence or absence of a condition, or may include numerical data to represent e.g. different classes. In contrast, “time-series data” is data which describes a change in a particular attribute with time. In the present invention, longitudinal data is used to refer to a combination of context data and time-series data.

It is preferable that the context data is clinical context data. The clinical context data may comprise measurements of the values of one or more medical parameters, or values indicative of the presence or absence of e.g. a disease or a condition, or other medical indicator. The clinical context data may also include, for example, values of medical scores indicative of various medical issues. This allows the synthetic longitudinal data to be used to implement survival analysis in a meaningful clinical context.

The training data may comprise one or more tensors. Specifically, the context data may include, or be in the form of, a context tensor. The added noise may be in the form of a noise tensor. The time-series data may be in the form of a time-series tensor. Preferably, the context data and the added noise data are concatenable. However, in some cases, this may not be the case. The computer-implemented method may further comprise a step of receiving the context data and noise data representing the noise; and applying a linear transform either to the context data or the noise data, the linear transform configured to modify the context data or the noise data such that it is concatenable with the other of the context data or the noise data. Thus, application of the linear transform may give rise to linearly transformed context data or linearly transformed noise data. The computer-implemented method may then comprise concatenating the linearly transformed context data with the noise data, or the linearly transformed noise data with the context data, in both cases generating a concatenation. The concatenation may then form the training input to the TTS-GAN generator. In some cases, the concatenation may undergo additional transformation before forming the training input to the TTS-GAN generator, for example, the computer-implemented method may further comprise applying a linear transform to the concatenation, to generate a linearly transformed concatenation, wherein the linearly transformed concatenation forms the training input to the TTS-GAN generator.

We now discuss the structure of the TTS-GAN in more detail. As discussed, it comprises a TTS-GAN generator and a TTS-GAN discriminator, each of which is based on transformer encoder architecture. A transformer is a deep learning model that adopts the mechanism of self-attention, differentially weighting the significance of each part of the input data. Unlike e.g. recurrent neural networks, which process sequential input data, transformers process all of the input data at once, thereby providing context for any position in the input sequence. Transformers typically employ an encoder-decoder architecture. The encoder typically includes encoding layers that process the input iteratively one layer after another, while the decoder consists of decoding layers that do the same thing to the encoder's output. The function of each encoder layer is generally to generate encodings that contain information about which parts of the inputs are relevant to each other. It may then pass its encodings to the next encoder layer as inputs. Each decoder layer is preferably configured to do the opposite, taking all the encodings and using their incorporated contextual information to generate an output sequence. To achieve this, each encoder and decoder layer makes use of an attention mechanism. For each input, attention weighs the relevance of every other input and draws from them to produce the output. Each decoder layer has an additional attention mechanism that draws information from the outputs of previous decoders, before the decoder layer draws information from the encodings. Both the encoder and decoder layers may have a feed-forward neural network for additional processing of the outputs and may contain residual connections and/or layer normalization steps.

We now turn back to the structure of the TTS-GAN generator of the present invention in more detail. The TTS-GAN generator may comprise one or more compound generator units, each compound generator unit comprising a self-attention mechanism, and a feed-forward layer. The self-attention mechanism may comprise a multi-head attention block.

In the present context, the self-attention mechanism may be configured to compare all input sequence members with each other, in order to draw global dependencies between the input sequences. Alternatively put, the self-attention mechanism differentiably key-value searches the input sequences for each input, and adds the results to the output sequence. Such self-attention mechanisms (including multi-head self-attention layers), and their uses in transformer architecture are known^{2 3 4 5}. ²https://towardsdatascience.com/illustrated-self-attention-2d627e33b20a³https://data-science-blog.com/blog/2021/04/07/multi-head-attention-mechanism/⁴https://vaclavkosar.com/ml/transformers-self-attention-mechanism-simplified⁵https://doi.org/10.48550/arXiv.1706.03762

The purpose of the feed-forward layer is to convert the output of the self-attention mechanism layer into a form which can form an input to a subsequent self-attention mechanism layer. Alternatively put, the feed-forward layer has a similar effect to a cross-attention layer with a trainable embedding sequence.

Each compound generator unit may further comprise a normalization layer and a dropout layer surrounding the self-attention mechanism. Furthermore, each compound generator unit may comprise a normalization layer and a dropout layer surrounding the feed-forward layer. The normalization layers are preferably configured to the activations of the various nodes within a layer of the compound generator unit using the mean and standard deviation of the activation values. Preferably, the normalization layers are layer normalization layers⁶. Specifically, the layer normalization statistics over the hidden units are calculated in the same way, as follows: ⁶https://doi.org/10.48550/arXiv.1607.06450

$${μ}^{ι} = \frac{1}{H} {\sum }_{i = 1}^{H} {a}_{i}^{ι}$$

$${σ}^{ι} = \sqrt{\frac{1}{H} {\sum }_{i = 1}^{H} {( {a}_{i}^{ι} - {μ}^{ι} )}^{2}}$$

In which:
μ^{l} is the mean and σ^{l} is the standard deviation
αᵢ are the activation values
H is the number of hidden units.

A “hidden unit”, in this context refers to a component including layers of processors between the input and output layers.

In the dropout layer, various nodes or neurons within a given layer are nullified, or their weights are set to zero. In some cases, the weights of the remaining neurons may be scaled up such that the sum over all inputs remains unchanged. This helps to prevent overfitting during training.

The TTS-GAN generator may further comprise a positional embedding unit. The TTS-GAN encoder is configured to map the concatenation of the context data and noise data to a sequence. In order to generate such a sequence, it is necessary to divide the input data into a plurality of patches, and mark each patch with its position in the sequence. This is executed by the positional embedding unit. See section 3.2 of Li et al. (2022), cited earlier in this patent application.

The TTS-GAN generator may further comprise a convolutional layer configured to receive the output of the final compound generator unit, and to reduce the number of dimensions. Effectively, the convolutional layer, which may be, for example, a Conv2D layer, may be configured to reduce the data from the number of hidden dimensions to the number of real dimensions . . . .

We now discuss the structure of the TTS-GAN discriminator in more detail. As mentioned previously, the TTS-GAN discriminator may also be based on a transformer encoder architecture. Accordingly, the TTS-GAN discriminator may comprise one or more compound discriminator units, each generator unit comprising a self-attention mechanism, such as a multi-head attention block, and a feed-forward layer. The functions of these layers are the same as in the TTS-GAN generator.0

Each compound discriminator unit may further comprise a normalization layer and a dropout layer surrounding the self-attention mechanism. Furthermore, each compound discriminator unit may comprise a normalization layer and a dropout layer surrounding the feed-forward layer.

Furthermore, the TTS-GAN discriminator further comprises a positional embedding layer configured to receive the time-series data and the synthetic time-series data generated by the TTS-GAN generator and to execute the same function as in the generator.

The TTS-GAN discriminator may further comprise a classification head configured to receive the output from the final compound discriminator unit, and to classify the input as either real time-series data or synthetic time-series data.

As we will explain shortly, the only part of the TTS-GAN which is required for generation of synthetic longitudinal data is the TTS-GAN generator. In view of that, after training the TTS-GAN, specifically the TTS-GAN generator thereof, the computer-implemented method may further comprise discarding the TTS-GAN discriminator, to retain only the TTS-GAN generator.

The preceding disclosure explains how a suitable TTS-GAN generator may be generated. We now explain, in the context of the second aspect of the invention, how a suitably trained TTS-GAN generator may be used to generate high-quality synthetic longitudinal data. The second aspect of the present invention provides a computer-implemented method of generating synthetic longitudinal data for use in survival analysis, or for use in a clinical trial, or for use in clinical research, using a generator of a TTS-GAN, the computer-implemented method comprising: receiving input data, the input data comprising context data; applying a machine-learning model to the context data to generate synthetic context data having statistical properties corresponding to the statistical properties of the received context data; applying a trained TTS-GAN generator to the generated synthetic context data, the TTS-GAN generator configured to generate synthetic time-series data based on the synthetic context data. Preferably, the TTS-GAN generator has been generated according to the computer-implemented method of the first aspect of the invention. The computer-implemented method may comprise applying the TTS-GAN generator to the generated synthetic context data and noise data. That is, the input of the TTS-GAN generator may comprise synthetic context data and noise data. Specifically, the computer-implemented method may comprise receiving the synthetic context data and noise data; and applying a linear transform to either the context data or the noise data, the linear transform configured to modify the received synthetic context data or the noise data such that it is concatenable with the other of the synthetic context data or the noise data. Thus, application of the linear transform may give rise to linearly transformed synthetic context data or linearly transformed noise data. The computer-implemented method may then comprise concatenating the linearly transformed synthetic context data with the noise data, or the linearly transformed noise data with the context data, in both cases generating a concatenation. The concatenation may then form the input to the TTS-GAN generator. In some cases, the concatenation may undergo additional transformation before forming the training input to the TTS-GAN generator, for example, the computer-implemented method may further comprise applying a linear transform to the concatenation, to generate a linearly transformed concatenation, wherein the linearly transformed concatenation forms the input to the TTS-GAN generator.

According to the computer-implemented method of the second aspect of the invention, the machine-learning model is able to generate synthetic context data based on the input context data, and the trained TTS-GAN generator is then able to generate synthetic time-series data based on the synthetic context data. It will be readily appreciated that this enables the generation of large volumes of high-quality data.

The computer-implemented method of the second aspect of the invention may generate synthetic longitudinal data for use in a clinical trial. Thus, according to the present invention, longitudinal data can be generated for use in clinical trials in such a way that it is sufficiently similar to real (i.e., non-synthetic) longitudinal data to be usable in the clinical trial, while being anonymised. In this way, clinical trial requirements of sufficient privacy of data as well as sufficient accuracy of data may both be met.

As an example, the computer-implemented method of the second aspect of the invention may generate synthetic longitudinal data for use in a control arm of a clinical trial. A control arm may refer to an arm in which participants receive no intervention or treatment, or receive standard-of-care treatment. Thus, the synthetic context data and/or or the input context data may include data indicating the absence of a given intervention or treatment or standard-of-care treatment. The second aspect of the invention may advantageously allow that data for use in a control arm of a clinical trial can be generated from pre-existing real longitudinal data, because this pre-existing real longitudinal data can be anonymised into synthetic longitudinal data. Thus, there may be no need to enrol control participants in the clinical trial, as the data for use in the control arm can be obtained from pre-existing real data, and privacy requirements can still be met. In this way, the efficiency of clinical trials may be greatly improved. Alternatively, the synthetic longitudinal data may be used to augment real longitudinal data from control participants in the clinical trial. In this way, less participants may be required for the clinical trial, so the efficiency of the clinical trial may be greatly improved, and/or the variability of data used in the clinical trial may be enhanced. Further, generating the synthetic longitudinal data from pre-existing real longitudinal data may provide that much more data is readily available for use in the control arm of the clinical trial, and thus the accuracy of the clinical trial may be improved.

The computer-implemented method of the second aspect of the invention may generate synthetic longitudinal data for use in an intervention arm of a clinical trial. The synthetic longitudinal data may be used to augment real longitudinal data from participants in the intervention arm of the clinical trial. In this way, less participants may be required for the clinical trial, so the efficiency of the clinical trial may be greatly improved, and/or the variability of data used in the clinical trial may be enhanced. An intervention arm may refer to an arm in which participants receive a treatment, such as a drug. Thus, the synthetic context data and/or or the input context data may include data indicating the provision of a given intervention or treatment.

Accordingly, when used in the context of a clinical trial, the computer-implemented method of the second aspect of the invention may be used in a computer-implemented method for selecting a drug for further analysis, as is described below in more detail with reference to the fourth aspect of the invention.

It is preferable that the TTS-GAN generator has been trained to generate the synthetic time-series data based on a known relationship between the context data and time-series data. In view of that, the context data of the input data is preferably the same as the context data of the training data (i.e. the data used to train the TTS-GAN generator, e.g. according to the computer-implemented method of the first aspect of the invention).

After the synthetic context data and synthetic time-series data have been generated, the computer-implemented method may further comprise a step of aggregated the synthetic context data and the synthetic time-series data to generate synthetic longitudinal data. This step enables the combination of the generated synthetic data into one aggregated set of data, which may subsequently be used in survival analysis, or in the clinical trial or clinical research.

The machine-learning model which is used to generate the synthetic context data based on the input context data may take many forms. Indeed, various machine-learning models are available which are able to generate synthetic tabular data. However, in preferred implementations, the machine-learning model may be a generative adversarial network (GAN). It is important to stress that this GAN is entirely separate from the TTS-GAN which is employed to generate the synthetic time-series data. The GAN does not need to be able to generate synthetic time-series data, its role in the context of the present invention is only to generate synthetic context data, which does not contain any time-dependent elements. The GAN is preferably a conditional generative adversarial network (CTGAN). A CTGAN is a type of GAN which may be configured to generate synthetic context data based on real context data. The GAN or CTGAN has preferably been trained using real context data. For example, the GAN or CTGAN may have been trained according to the following computer-implemented method: receiving training context data in tabular form, the training data comprising column labels each defining a respective attribute of an entity, and records defining the values of the attribute for each of a respective plurality of entities; and training the GAN using the training context data, thereby generating a trained GAN configured to generate synthetic context data having statistical properties corresponding to the statistical properties of the training context data. The statistical properties in question may comprise one or more of the mean, and standard deviation of the received context data. Preferably the statistical properties of the synthetic context data are equal to, or approximately equal to, the statistical properties of the received context data.

The GAN may comprise a generator and a discriminator. Generally, the generator is trained to generate more and more convincing synthetic context data, and the discriminator is concurrently trained to discriminate more and more effectively between the real context data and the synthetic context data. This process is repeated iteratively until a predetermined end condition is met. The difference between the synthetic context data generated by the generator and the real context data is parameterized in the form of a generator loss function. Correspondingly, the mistakes made by the generator (i.e. by characterizing the real context data as synthetic context data, and vice versa) are parameterized in the form of a discriminator loss function. Thus, training the GAN may comprise: (a) training the generator to minimize a generator loss function; and (b) training the discriminator to minimize a discriminator loss function, wherein steps (a) and (b) are repeated alternately until a predetermined end condition is met.

Loss functions may take many forms. In Goodfellow et al. (2014)⁷, a minimax loss function is formulated, which the generator aims to minimize, while the discriminator aims to maximize it. ⁷https://doi.org/10.48550/arXiv.1406.2661

$${E}_{x} \cdot log ( D ( x ) ) + {E}_{z} \cdot log ( 1 - D ( G ( z ) ) )$$

In this function:
D(x) is the discriminator's estimate of the probability that real data instance x is real.
Eₓ is the expected value over all real data instances.
G(z) is the generator's output when given noise z.
D(G(z)) is the discriminator's estimate of the probability that a fake instance is real.
E_{z} is the expected value over all random inputs to the generator (in effect, the expected value over all generated fake instances G(z)).

The generator cannot directly affect the log D(x) term, so for the generator, minimizing the loss is equivalent to minimizing log (1−D(G(z))). In some cases, the loss function may be modified such that the generator aims to maximize log D(G(z)).

In other cases, Wasserstein Gradient Penalty Loss⁸ may be used for the GANs. The WGAN-GP loss augments Wasserstein loss with gradient norm penalty for random samples \hat{x}˜P_{\hat{x}}. ⁸https://doi.org/10.48550/arXiv.1704.00028

$$L = {E}_{\hat{x} - {P}_{g}} [ D ( \tilde{x} ) ] - {E}_{x ∼ {P}_{r}} [ D ( x ) ] + λ {E}_{\hat{x} ∼ {P}_{\hat{x}}} [ {( {\vert \vert {∇}_{\tilde{x}} D ( \tilde{x} ) \vert \vert}_{2} - 1 )}^{2} ]$$

In another implementation, the following loss functions may be used:

$${L}_{D} = - {L}_{adv} + λ {L}_{c l s}^{r}$$

$${L}_{G} = {L}_{adv} + λ {L}_{c l s}^{f}$$

Real context data necessarily comprises information about real individuals. In the context of e.g. survival analysis, in which medical data may be used, it is important to ensure that it is not possible to identify specific individuals from the real context data. Breaches of privacy such as this must be avoided. In the present invention, this can be achieved by incorporating a differential privacy component into the discriminator loss function. Specifically, training the GAN comprises adding a differential privacy component e to the discriminator loss function. The differential privacy component e parameterizes the privacy loss when one entry is added to or removed from the training context data.

In some cases, adding the differential privacy component may comprise adding random noise to the discriminator loss function.

Intuitively, a model trained using such a method should not be affected by adding or removing a single training example. In this way, it is possible to ensure that the synthetic context data, and the synthetic time-series data generated therefrom do not enable third parties to identify or work out the identities of individual patients whose context data was used to generate the synthetic longitudinal data.

The generator of the GAN may comprise one or more residual layers and one or more linear layers. The discriminator may comprise one or more linear layers, one or more rectified linear unit (ReLu) layers, and one or more dropout layers.

As we have discussed, the combination of the first and second aspects of the invention act to produce high-quality synthetic longitudinal data for use in survival analysis. Further aspects of the invention are directed towards the use of the synthetic longitudinal data in survival analysis. A prime use of survival analysis is the prediction of the point in time at which a given event takes place. Such an event could be a biological or a clinical event (e.g. death, onset of a disease, organ failure), or an engineering event (e.g. failure of a component), or the like. Accordingly, a third aspect of the invention provides a computer-implemented method of executing survival analysis to predict a time at which a predetermined event will occur, or an amount of time remaining until the predetermined event will occur, the computer-implemented method comprising: receiving context data defining the values of a plurality of attributes for an entity; generating (e.g. using the computer-implemented method of the second aspect of the invention) synthetic longitudinal data representative of the change in value of one or more predetermined variables; and determining, based on the generated synthetic longitudinal data, the point in time at which the predetermined event will occur, or the amount of time remaining until the predetermined event will occur. It is evident that the present invention is particularly advantageous for this kind of analysis, since it enables the generation of high-quality synthetic longitudinal data which represents the likely time-series progression of a variable, based only on context data received about a particular entity.

The survival analysis may be a clinical survival analysis, which may estimate the point in time at which a predetermined clinical event will occur, or the amount of time remaining until the predetermined clinical event will occur. In this way, the third aspect of the present invention may be used to generate clinical survival analysis data for use in a clinical trial. Thus, the third aspect of the invention may provide a computer-implemented method of generating clinical survival analysis data for use in a clinical trial. Clinical survival analysis data may refer to the point in time at which the predetermined clinical event will occur, or the amount of time remaining until the predetermined clinical event will occur. Similar to the advantages discussed above with reference to the second aspect of the invention, there may be several advantages to using clinical survival analysis data which is generated from synthetic longitudinal data in a clinical trial. For example, the clinical survival analysis data may be generated from longitudinal data which is sufficiently similar to real (i.e., non-synthetic) longitudinal data to be usable in the clinical trial, while being anonymised. Thus, the clinical trial requirements of sufficient privacy of data as well as sufficient accuracy of data may both be met.

As an example, the third aspect of the invention may provide a computer-implemented method of generating clinical survival analysis data for use in a control arm of a clinical trial. The clinical survival analysis data may be used to augment real clinical survival analysis data from participants in the control arm of the clinical trial. The synthetic context data, from which the synthetic time-series data and thus the clinical survival analysis data is generated, may include data indicating the absence of a given intervention or treatment. The input context data, from which the synthetic context data is generated, may include data indicating the absence of a given intervention or treatment. In this way, the clinical survival analysis data for use in the control arm of a clinical trial can be generated from pre-existing real longitudinal data, because this pre-existing real longitudinal data can be anonymised into synthetic longitudinal data. Thus, there may be no need to enrol control participants in the clinical trial, as the data for use in the control arm can be obtained from pre-existing real data, and privacy requirements can still be met. In this way, the efficiency of clinical trials may be greatly improved. Alternatively, the clinical survival analysis data may be used to augment real survival analysis data from control participants in the clinical trial. In this way, less participants may be required for the clinical trial, so the efficiency of the clinical trial may be greatly improved, and/or the variability of data used in the clinical trial may be enhanced. Further, generating the clinical survival analysis data from synthetic longitudinal data, in turn based on pre-existing real longitudinal data, may provide that much more data is readily available for use in the control arm of the clinical trial, and thus the accuracy of the clinical trial may be improved.

The third aspect of the invention may provide a computer-implemented method of generating clinical survival analysis data for use in an intervention arm of a clinical trial. The clinical survival analysis data may be used to augment real clinical survival analysis data from participants in the intervention arm of the clinical trial. In this way, less participants may be required for the clinical trial, so the efficiency of the clinical trial may be greatly improved, and/or the variability of data used in the clinical trial may be enhanced. The synthetic context data, from which the synthetic time-series data and thus the clinical survival analysis data is generated, may include data indicating the provision of a given intervention or treatment. The input context data, from which the synthetic context data is generated may include data indicating the provision of a given intervention or treatment. Accordingly, the third aspect of the invention may be used in a computer-implemented method for selecting a drug for further analysis, as is described below in more detail with reference to the fifth aspect.

Determining the point in time or the amount of time remaining may comprise, based on the synthetic longitudinal data, generating a plot of the probability of the predetermining event occurring against time. Then, determining the point in time or the amount of time remaining may comprise determining the point in time at which the probability of the predetermined event occurring is greater than or equal to a predetermined threshold. Where necessary, the amount of time remaining until the determined time may then be calculated.

The plot of the probability against time may be a Kaplan-Meier curve⁹. ⁹https://en.wikipedia.org/wiki/Kaplan % E2%80%93Meier_estimator

In survival analysis, the probability of an event happening at a certain time point may be given using:

$$\hat{S} ( t ) = {\prod }_{i : {t}_{i} \leq t} ( 1 - \frac{{d}_{i}}{{n}_{i}} )$$

Herein:
tᵢ is the time at which at least one event happened.
dᵢ is the number of events (e.g. deaths) that happened at time tᵢ.
nᵢ is the number of “non-events” (e.g. the number of individuals known to have survived), i.e. individuals or entities for whom the event has not yet taken place.

Examples of the predetermined event may include death (e.g. after a diagnosis of a given disease, or after a given treatment or regimen of treatment has been administered), onset of a disease, onset of a symptom, relapse of a condition such as MS or cancer, metastasis, or organ failure. In these cases, it is preferable that the context data is clinical context data. The clinical context data may comprise measurements of the values of one or more medical parameters, or values indicative of the presence or absence of e.g. a disease or a condition, or other medical indicator. The clinical context data may also include, for example, values of medical scores indicative of various medical issues. This allows the synthetic longitudinal data to be used to implement survival analysis in a meaningful clinical context.

As discussed, survival analysis may also be used in an engineering context, in which case the predetermined event may be failure of a component.

As discussed, synthetic longitudinal data and/or clinical survival analysis data, generated from synthetic longitudinal data, may be used in intervention arm of clinical trials. As further discussed, in these cases the synthetic context data and/or the input context data may include an indication that (respectively synthetic or real) participants have received a given intervention or treatment, such as a drug. Using the synthetic longitudinal data and/or clinical survival analysis data, a drug may be selected for further development. For example, a drug associated with desirable synthetic time-series data and/or desirable clinical survival analysis data may be selected for further development.

Accordingly, a fourth aspect of the invention may provide a computer-implemented method of selecting a drug for further development, the computer-implemented method comprising: generating, according to the second aspect of the present invention, synthetic longitudinal data for use in a clinical trial, wherein a portion of the synthetic context data includes data indicating the provision of a given drug; receiving real longitudinal data for use in the clinical trial, the real longitudinal data comprising real context data and real time-series data and being acquired from a plurality of participants provided with the given drug; determining whether time-series data comprising a portion of the synthetic time-series data and comprising the real time-series data meets a predetermined criterion, the portion of the synthetic time-series data generated from the portion of the synthetic context data; and, in response to a determination that the time-series data meets the predetermined criterion, selecting the given drug for further analysis and/or development. The criterion may be indicative of some favourable condition which may be, for example, a fast improvement of a particular attribute, or a slow deterioration of a particular attribute.

The portion of the synthetic context data may be the only synthetic context data used to generate the portion of the synthetic time-series data.

Similarly, a fifth aspect of the invention may provide a computer-implemented method of selecting a drug for further development, the computer-implemented method comprising: generating, according to the third aspect of the present invention, clinical survival analysis data for use in a clinical trial, wherein a portion of the synthetic longitudinal data includes data indicating the provision of a given drug; receiving real clinical survival analysis data for use in the clinical trial, the real clinical survival analysis data being acquired from participants provided with the given drug; determining whether clinical survival analysis data, comprising a portion of the synthetic clinical survival analysis data and comprising the real clinical survival analysis data, meets a predetermined criterion, the portion of the clinical survival analysis data generated from the portion of the synthetic longitudinal data; and, in response to a determination that the clinical survival analysis data meets the predetermined criterion, selecting the given drug for further analysis and/or development. The criterion may comprise a favourable condition which may be, for example, a distant point in time at which a harmful or negative clinical event will occur, or a long amount of time until a harmful or negative clinical event will occur. The favourable condition may be, for example, a near point in time at which a beneficial or positive clinical event will occur, or a short amount of time until a beneficial or positive clinical event will occur.

A portion of the synthetic longitudinal data including data indicating the provision of a given drug may refer to a portion of the synthetic context data including data indicating the provision of a given drug. The portion of the clinical survival analysis data being generated from the portion of the synthetic longitudinal data may refer to the portion of the clinical survival analysis data being generated from a portion of the synthetic time-series data, the portion of the synthetic time-series data being generated from the portion of the synthetic context data.

The portion of the synthetic longitudinal data may be the only synthetic longitudinal data used to generate the portion of the clinical survival analysis data. The portion of the synthetic context data may be the only synthetic context data used to generate the portion of the synthetic time-series data. The portion of the synthetic time series data may be the only synthetic time-series data used to generate the portion of the clinical survival analysis data.

Synthetic longitudinal data is useful in situations where large volumes of data is required, but such data is simply not available. This may be the case when training machine-learning models. Accordingly, a sixth aspect of the invention may provide a computer-implemented method of generating a trained machine-learning model, the computer-implemented method comprising: receiving longitudinal training data; generating synthetic longitudinal data based on the longitudinal training data using the computer-implemented method of the second aspect of the invention; and training a machine-learning model using the generated synthetic longitudinal data. The machine-learning model may be a deep learning model. The machine-learning model may be configured to generate a clinically meaningful output, and may for example be a clinical recommendation model, a clinical decision support model, or a predictive model.

Synthetic longitudinal data is useful in situations where anonymised data is required, because the synthetic longitudinal data is able to conceal the identities of patients whose context data was used to generate the synthetic longitudinal data to be identified. Data anonymisation may be required, for example, when data is being transmitted from a data source to a data consumer external to the data source. For example, a data source may be a hospital, and a data consumer may be a research institution. Accordingly, a seventh aspect of the invention may provide a computer-implemented method of generating synthetic longitudinal data, the computer-implemented method comprising: receiving a request for synthetic longitudinal data; applying a trained machine-learning model to input data comprising context data to generate synthetic context data having statistical properties corresponding to the statistical properties of the context data; applying a trained transformer-based time-series generative adversarial network (TTS-GAN) generator to the generated synthetic context data to generate synthetic time-series data based on the synthetic context data; and outputting synthetic longitudinal data comprising the synthetic context data and the synthetic time-series data. The computer-implemented method according to the seventh aspect may be carried out by a data source. One or more steps of the computer-implemented method may be carried out on a data source local server.

In this way, the present invention may provide a parallel technique to existing data anonymisation techniques. Data may be output which is sufficiently similar to real patient data to be suitable for research, for example, while meeting data privacy requirements.

It will be appreciated that the seventh aspect of the invention is a computer-implemented method which includes steps of the second aspect of the invention. Accordingly, the features set out above in respect of the second aspect of the invention may be included in the seventh aspect of the invention, except where clearly technically incompatible or where context dictates otherwise.

The trained TTS-GAN generator may be configured to generate synthetic time-series data based on input context data. The trained TTS-GAN generator may have been generated by training a TTS-GAN, the TTS-GAN comprising a TTS-GAN generator and a TTS-GAN discriminator. The trained TTS-GAN may have been trained using longitudinal training data comprising training context data and training time-series data. Training the TTS-GAN using the longitudinal training data may comprise: (a) training the TTS-GAN generator to generate synthetic time-series data using the training context data and added noise; (b) training the TTS-GAN discriminator to discriminate between the training time-series data and the synthetic time-series data generated by the TTS-GAN generator; (c) repeating steps (a) and (b) alternately until a predetermined end condition is met. Training the TTS-GAN may comprise generating, according to the first aspect of the present invention, a TTS-GAN configured to generate longitudinal synthetic data.

The computer-implemented method may further comprise receiving, or installing the transformer-based time-series generative adversarial network (TTS-GAN) comprising the TTS-GAN generator and the TTS-GAN discriminator. The TTS-GAN may be received from a data consumer local server. A data consumer remote server may store the TTS-GAN. A remote server may comprise a cloud server, for example. The computer-implemented method may comprise accessing the data consumer remote server. Accessing the data consumer remote server may comprise accessing the data consumer remote server by inputting a data source key. The data source key may be a unique user identification or a password. The TTS-GAN may be installed via a command line or a web-browser. The TTS-GAN may be received from the data consumer remote server. The steps of receiving or installing the TTS-GAN and/or accessing the data consumer remote server may be carried out by one or more data source local servers.

The computer-implemented method may further comprise training the TTS-GAN using the longitudinal training data to obtain the trained TTS-GAN generator. The TTS-GAN may be trained locally. For example, the TTS-GAN may be trained locally on a data source local server. Alternatively, the TTS-GAN may be trained remotely. For example, the TTS-GAN may be trained on a first data source remote server. The TTS-GAN may be trained in a federated learning environment. The computer-implemented method may comprise transmitting, for example by the data source local server, the TTS-GAN, and receiving, for example by the first data source remote server, the TTS-GAN. The computer-implemented method may comprise transmitting, for example by the first data source remote server, the trained TTS-GAN generator, and receiving, for example by the data source local server, the trained TTS-GAN generator.

The trained machine-learning model may be the machine-learning model described with reference to the second aspect of the invention. The trained machine-learning model may be a GAN, for example. The trained machine learning model may have been trained as described with reference to the first aspect of the invention.

The computer-implemented method may further comprise receiving, or installing a machine-learning model, and training the machine-learning model to obtain the trained machine-learning model. The machine-learning model may be received from the data consumer local server. A data consumer remote server may store the machine-learning model. The computer-implemented method may comprise accessing the data consumer remote server. Accessing the data consumer remote server may comprise accessing the data consumer remote server by inputting a data source key. The data source key may be a unique user identification or a password. The machine-learning model may be installed via a command line or a web-browser.

The machine-learning model may be received from the data consumer remote server.

The machine learning model may be trained locally. For example, the machine learning model may be trained locally on a data source local server. Alternatively, the machine learning model may be trained remotely. For example, the machine learning model may be trained on a first data source remote server. The machine learning model may be trained in a federated learning environment.

The request for synthetic longitudinal data may be received from a data consumer local server.

The computer-implemented method may comprise applying the TTS-GAN generator to the generated synthetic context data and noise data. That is, the input of the TTS-GAN generator may comprise synthetic context data and noise data.

The step of applying the TTS-GAN generator may be carried out at a data source local server. The step of applying the trained machine-learning model may be carried out at a data source local server.

Outputting the synthetic longitudinal data may include transmitting the synthetic longitudinal data, for example, transmitting the synthetic longitudinal data to the data consumer local server, or transmitting the synthetic longitudinal data to a second data source remote server. The step of outputting the synthetic longitudinal data may be carried out at a data source local server. The synthetic longitudinal data may be stored on the second data source remote server, and may be accessible on the second data source remote server by the data consumer local server. The computer-implemented method may further comprise providing access to the second data source remote server. Providing access to the second data source remote server may comprise receiving a key, and if the key matches a predetermined data consumer key, authorising access to the second data source remote server.

Outputting the synthetic longitudinal data may include transmitting the synthetic longitudinal data via a network.

As discussed, one or more steps of the computer-implemented method according to the seventh aspect may be carried out on one or more data source local servers. The longitudinal training data may be stored locally, and/or may be accessible, by the data source local server. The input data may be stored locally, and/or may be accessible, by the data source local server. The computer-implemented method may comprise receiving input data and/or the longitudinal training data. The input data and/or the longitudinal training data may be received from a local data store. The longitudinal training data may be stored remotely from, and/or may be inaccessible, by the data consumer. The input data may be stored remotely from, and/or may be inaccessible, by the data consumer. Therefore, the seventh aspect enables data to be used, for example for research purposes, without breaching data privacy requirements.

In some examples, the computer-implemented method may further comprise calculating and/or displaying a statistical metric of the synthetic longitudinal data based on a comparison of the synthetic longitudinal data and real longitudinal data of which the context data was used to generate the synthetic longitudinal data. The step of calculating the statistical metric may be carried out on a data source local server.

The statistical metric may include a privacy metric and/or a quality metric.

Calculating the quality metric may include one or more of: (a) conducting a univariate comparison; (b) conducting a comparison test; (c) conducting a machine-learning performance comparison; and (d) determining a distinguishability of the data sets.

Conducting the univariate comparison may comprise: for each longitudinal data variable calculating and/or displaying a similarity or a difference between the corresponding synthetic longitudinal data and real longitudinal data. A higher similarity may result in a higher quality metric. A higher difference may result in a lower quality metric. The similarity may correspond to the quality metric. Calculating the similarity or the difference may comprise comparing values of the mean, the standard deviation, the minimum value, and/or the maximum value. Calculating the similarity or the difference may comprise calculating a level of overlap, such as a percentage overlap, between the univariate distributions of the corresponding synthetic longitudinal data and the real longitudinal data. Calculating the similarity or the difference may comprise performing a t-test and/or a chi-squared test between the corresponding real longitudinal data and the synthetic longitudinal data. For example, a t-test may be performed for numerical variables. A chi-squared test may be performed for categorical variables. Displaying the similarity or the difference may comprise displaying a statistical property of the corresponding synthetic longitudinal data and real longitudinal data, such as the mean, the standard deviation, the minimum value and/or the maximum value. Displaying the similarity or the difference may comprise displaying a bar plot and/or a violin plot, and/or a density plot of the corresponding synthetic longitudinal data and/or the corresponding real longitudinal data.

Conducting the bivariate comparison may comprise: for a pair of longitudinal data variables, calculating a correlation of the corresponding synthetic longitudinal data, and calculating a correlation of the corresponding real longitudinal data; and calculating a similarity of or a difference between the correlations. Conducting the bivariate test may comprise: for every pair of longitudinal data variables, calculating a correlation of the corresponding synthetic longitudinal data, and calculating a correlation of the corresponding real longitudinal data; and calculating a similarity or a difference of the correlations. A higher similarity may result in a higher quality metric. A higher difference may result in a lower quality metric. The similarity may correspond to the quality metric. Calculating the correlations may comprise calculating Theils' U statistic, calculating Pearson's correlation coefficient and/or calculating a correlation ratio. Theils' U statistic may be calculated for a pair of longitudinal data variables which includes a categorical variable and a categorical variable. Pearson's correlation coefficient may be calculated for a pair of longitudinal data variables which includes a numerical variable and a numerical variable. The correlation ratio may be calculated for a pair of longitudinal data variables which includes a numerical variable and a categorical variable. Conducting the bivariate test may further comprise calculating and/or displaying a correlation matrix for the real longitudinal data and calculating and/or displaying a correlation matrix for the synthetic longitudinal data. Conducting the bivariate test may further comprise calculating an absolute difference, such as an absolute mean difference, between the correlation matrices. A higher absolute difference may result in a lower quality metric.

Conducting the machine-learning performance comparison may comprise: evaluating the performance of a machine-learning model trained using the real longitudinal data; evaluating the performance of a machine-learning model trained using the synthetic longitudinal data; and, using the evaluations, calculating or displaying a similarity or difference of the performances of the machine learning models. A higher similarity may result in a higher quality metric. A higher difference may result in a lower quality metric. The similarity may correspond to the quality metric. Before training, the machine-learning models to be trained using each data type may be the same. The same test may be used to evaluate the performance of each machine-learning model. Evaluating the performance of a machine-learning model may comprise evaluating the performance of the model at predicting a specific variable. Evaluating the performance of a machine-learning model may comprise obtaining a confusion matrix. Evaluating the performance of a machine-learning model may comprise calculating an F1 score. Displaying the similarity may comprise displaying the confusion matrix for each machine-learning model.

Determining the distinguishability of the data sets may comprise: combining the real longitudinal data and the synthetic longitudinal data; applying a distinguishing model to the combined real longitudinal data and synthetic longitudinal data; and, evaluating the performance of the distinguishing model. A higher performance of the distinguishing model may correspond to a lower quality metric. Combining the real longitudinal data may comprise shuffling real and synthetic data rows into one dataset. The distinguishing model may classify which rows correspond to synthetic longitudinal data and which rows correspond to real longitudinal data. Conducting the distinguishability test may comprise training the distinguishing model to distinguish between the real longitudinal data and the synthetic longitudinal data. Evaluating the performance of the distinguishing model may comprise obtaining a confusion matrix. Determining the distinguishability of the data sets may comprise displaying the confusion matrix. Evaluating the performance of the distinguishing model may comprise calculating an F1 score. An F1 score of 0.5 may correspond to an optimum quality score.

Calculating the privacy metric may include: evaluating the level of anonymisation of the synthetic data, or, in other words, evaluating the risk of re-identification of patients whose context data was used to generate the synthetic longitudinal data. Evaluating the level of anonymisation of the synthetic data may take into account attribute criteria stated in the GDPR, and in particular, may take into account inference, singling out and/or linkability.

If the calculated statistical metric indicates that the statistical (e.g., the privacy and/or the quality) of the synthetic longitudinal data is insufficient, the TTS-GAN may be re-trained and the synthetic longitudinal data may be re-generated. That is, the computer-implemented method may further comprise: determining whether the statistical metric meets a predetermined threshold; and, in response to determining that the statistical metric does not meet a predetermined threshold, adjusting a value of a training parameter of the TTS-GAN and re-training the TTS-GAN using the adjusted training parameter value and longitudinal training data, which may be the same as or different from the longitudinal training data used in the original training. These steps may be performed iteratively until it is determined that the statistical metric meets the predetermined threshold. In this way, it may be ensured that only data with sufficient privacy and/or quality is transmitted and/or accessible.

The training parameter may include a batch size, a learning rate, an objective function, a number of layers/neurons, a noise vector, a weight-clipping, a dropout, a weight-regularization, a number of epochs or a number of discriminator steps per generator step. Thus, the training parameter may include the added noise. Smaller batch sizes may help improve generalization and avoid overfitting to the training data, enhancing privacy. But, a batch size which is too small may damage model quality. A properly tuned learning rate may be important for model convergence and quality. A learning rate that which is too high may cause the generator to overfit to the discriminator, reducing training data privacy. Changing the objective function may improve privacy or quality depending on needs. More layers and neurons may increase model capacity and quality, but may also increase overfitting, potentially exposing private attributes in the training data. Simpler models may provide better privacy. The initial random noise vectors fed into the generator may affect quality and overfitting. More noise may make the mapping harder to learn, but may enhance privacy. Weight clipping may reduce influence of individual data points (making learning more stable, less likely to overfit to a specific person). Adding dropout to the discriminator and the generator may help to prevent overfitting, improving privacy. But too much dropout may reduce model quality. Methods like L1/L2 regularization may discourage large weights, reducing overfitting and improving privacy. But too strong regularization may harm quality. Training for more epochs may improve model convergence but may also lead to overfitting, providing less privacy. Early stopping may help. Training the discriminator more before each generator update may improve quality, but may also allow discriminator overfitting, hurting privacy.

For the avoidance of doubt, re-training the TTS-GAN using the longitudinal training data may comprise: (a) training the TTS-GAN generator to generate synthetic time-series data using the training context data and added noise; (b) training the TTS-GAN discriminator to discriminate between the training time-series data and the synthetic time-series data generated by the TTS-GAN generator; (c) repeating steps (a) and (b) alternately until a predetermined end condition is met. The computer-implemented method may further comprise applying the re-trained TTS-GAN to the generated synthetic context data to generate new synthetic time-series data.

An eighth aspect of the invention may provide a computer-implemented method of receiving synthetic longitudinal data, the computer-implemented method comprising: transmitting a request for synthetic longitudinal data; transmitting a transformer-based time-series generative adversarial network (TTS-GAN) comprising a TTS-GAN generator and a TTS-GAN discriminator, the TTS-GAN configured to be trained using longitudinal training data comprising training context data and training time-series data by: (a) training the TTS-GAN generator to generate synthetic time-series data using the training context data and added noise; (b) training the TTS-GAN discriminator to discriminate between the training time-series data and the synthetic time-series data generated by the TTS-GAN generator; (c) repeating steps (a) and (b) alternately until a predetermined end condition is met; to generate a trained TTS-GAN generator configured to generate synthetic time-series data based on input data comprising context data; and, receiving synthetic longitudinal data comprising synthetic context data and synthetic time series data, wherein the synthetic context data has been generated by applying a trained machine-learning model to input data comprising context data to generate synthetic context data having statistical properties corresponding to the statistical properties of the context data, and wherein the synthetic time-series data has been generated by the trained TTS-GAN generator. The computer-implemented method according to the eighth aspect may be carried out by a data consumer. One or more steps of the computer-implemented method according to the eighth aspect may be carried out on one or more data consumer local servers.

The request may be transmitted to a data source local server. The step of transmitting the request may be carried out by the data consumer local server.

The TTS-GAN may be transmitted to the data source local server. The TTS-GAN may be transmitted to a data consumer remote server. The step of transmitting the TTS-GAN may be carried out by a data consumer local server. The data consumer remote server may be external to the data source. The data consumer remote server may be external to the data consumer. The TTS-GAN may be stored on the data consumer remote server and may be accessible on the data consumer remote server by the data source. The computer-implemented method may further comprise providing access to the data consumer remote server. Providing access to the data consumer remote server may comprise receiving a key, and if the key matches a predetermined data source key, authorising access to the second data source remote server.

The synthetic longitudinal data may be received from a data source local server. A second data source remote server may store the synthetic longitudinal data. The computer-implemented method may comprise accessing the second data source remote server. Accessing the second data source remote server may comprise accessing the second data source remote server by inputting a data consumer key. The data consumer key may be a unique user identification or a password. The synthetic longitudinal data may be received from the second data source remote server. The step of receiving the synthetic longitudinal data and/or accessing the second data source remote server may be carried out by a data consumer local server.

The computer-implemented method may further comprise transmitting a machine-learning model configured to be trained to become the trained machine-learning model, the trained machine-learning model configured to generate synthetic context data based on input data comprising context data, the synthetic context data having statistical properties corresponding to the statistical properties of the context data. The machine-learning model may be transmitted to a data source local server. The machine-learning model may be transmitted to a data consumer remote server. The step of transmitting the machine-learning model may be carried out by a data consumer local server. The data consumer remote server may be external to the data source. The data consumer remote server may be external to the data consumer. The machine-learning model may be stored on the data consumer remote server and may be accessible on the data consumer remote server by the data source. The computer-implemented method may further comprise providing access to the data consumer remote server. Providing access to the data consumer remote server may comprise receiving a key, and if the key matches a predetermined data source key, authorising access to the data consumer remote server.

Transmitting the request, the TTS-GAN and/or the machine learning model may include transmitting via a network the request, the TTS-GAN, and/or the machine learning model.

One or more steps of the computer-implemented method according to the eighth aspect may be carried out by one or more data consumer local servers.

A computer-implemented method according to a ninth aspect of the present invention may provide a combination of the computer-implemented methods according to the seventh and eighth aspects of the present invention, which may be carried out respectively by a data source and a data consumer. That is, a computer-implemented method according to a ninth aspect of the present invention may provide: a computer-implemented method of exchanging synthetic longitudinal data for use in clinical research, the computer-implemented method comprising: transmitting a request for synthetic longitudinal data; receiving a request for synthetic longitudinal data; transmitting a transformer-based time-series generative adversarial network (TTS-GAN) comprising a TTS-GAN generator and a TTS-GAN discriminator; receiving the TTS-GAN; training the TTS-GAN, using longitudinal training data comprising training context data and training time-series data, wherein training the TTS-GAN comprises: (a) training the TTS-GAN generator to generate synthetic time-series data using the training context data and added noise; (b) training the TTS-GAN discriminator to discriminate between the training time-series data and the synthetic time-series data generated by the TTS-GAN generator; (c) repeating steps (a) and (b) alternately until a predetermined end condition is met; applying a trained machine-learning model to input data comprising context data to generate synthetic context data having statistical properties corresponding to the statistical properties of the context data; applying the trained transformer-based time-series generative adversarial network (TTS-GAN) generator to the generated synthetic context data to generate synthetic time-series data based on the synthetic context data; transmitting synthetic longitudinal data comprising the synthetic context data and the synthetic time-series data; and, receiving the synthetic longitudinal data.

It will be appreciated that the features set out above in respect of the seventh and eighth aspects of the invention may be included in the ninth aspect of the invention, except where clearly technically incompatible or where context dictates otherwise.

The preceding disclosure relates to computer-implemented methods. It should be noted that the computer-implemented methods of the first, second, third, fourth, fifth, sixth, seventh, eighth and/or ninth aspects of the invention may be combined.

Additional aspects of the invention may relate to e.g. computer programs, and systems.

For example, a tenth aspect of the invention may provide a computer program which when run by a computer causes the computer, or a processor thereof, to execute the computer-implemented method of one or more of the first, second, third, fourth, fifth, sixth, seventh, eighth and ninth aspects of the invention. An eleventh aspect of the invention may provide a computer-readable medium storing the computer program of the tenth aspect of the invention.

A twelfth aspect of the invention may provide a TTS-GAN generation module configured to generate a transformer-based time-series generative adversarial network (TTS-GAN) configured to generate longitudinal synthetic data for use in survival analysis, the TTS-GAN comprising a TTS-GAN generator and a TTS-GAN discriminator, the TTS-GAN generation module comprising: an interface module configured to receive longitudinal training data comprising context data and time-series data; a training module configured to train the TTS-GAN using the longitudinal training data, the training module comprising a TTS-GAN generator optimizer and a TTS-GAN discriminator optimizer, wherein: (a) the TTS-GAN generator optimizer is configured to train the TTS-GAN generator to generate synthetic time-series data using the context data and added noise; (b) the TTS-GAN discriminator optimizer is configured to train the TTS-GAN discriminator to discriminate between the time-series data and the synthetic time-series data generated by the TTS-GAN generator; and (c) the training module is configured to alternate the operations executed in (a) and (b) until a predetermined end condition is met. The training module may further comprise an output module which is configured to output and or store the trained TTS-GAN after the predetermined end condition is met. It will be appreciated that the twelfth aspect of the invention is a system which is adapted to execute the computer-implemented method of the first aspect of the invention. Accordingly, the optional features set out above in respect of the first aspect of the invention also apply equally well to the twelfth aspect of the invention, except where clearly technically incompatible or where context dictates otherwise.

Analogous to the second aspect of the invention, a twelfth aspect of the invention provides a synthetic longitudinal data generation module configured to generate synthetic longitudinal data for use in survival analysis, the synthetic longitudinal data generation module comprising: an interface module configured to receive input data, the input data comprising context data; a synthetic context data generation module configured to apply a machine-learning model to the context data to generate synthetic context data having statistical properties corresponding to the statistical properties of the received context data; a synthetic time-series data generation module configured to apply a trained TTS-GAN generator to the generated synthetic context data to generate synthetic time-series data based on the synthetic context data. The synthetic longitudinal data generation module may further comprise an aggregation module configured to aggregate the synthetic context data and the synthetic time-series data to generate the synthetic longitudinal data. Preferably, the TTS-GAN generator is generated using the TTS-GAN generation module of the twelfth aspect of the invention. In which case, the interface module may further be configured to receive the TTS-GAN (or at least the generator thereof) before applying it to the synthetic context data. It will be appreciated that the thirteenth aspect of the invention is a system which is adapted to execute the computer-implemented method of the second aspect of the invention. Accordingly, the optional features set out above in respect of the second aspect of the invention also apply equally well to the thirteenth aspect of the invention, except where clearly technically incompatible or where context dictates otherwise.

Analogous to the third aspect of the invention, a fourteenth aspect of the invention may provide a survival analysis module configured to predict a time at which a predetermined event will occur, or an amount of time remaining until the predetermined event will occur, the survival analysis module comprising: an interface module configured to receive context data defining the values of a plurality of attributes for an entity; a synthetic longitudinal data module configured to generate or receive synthetic longitudinal data representative of the change in value of one or more predetermined variables; and a prediction module configured to determine, based on the generated synthetic longitudinal data, the point in time at which the predetermined event will occur, or the amount of time remaining until the predetermined event will occur. The synthetic longitudinal data module is preferably either the synthetic longitudinal data generation module of the thirteenth aspect of the invention, or is configured to receive synthetic longitudinal data generated by that module. The survival analysis module may further comprise a plot generation module configured to generate, based on the synthetic longitudinal data, generating a plot of the probability of the predetermining event occurring against time. It will be appreciated that the fourteenth aspect of the invention is a system which is adapted to execute the computer-implemented method of the third aspect of the invention. Accordingly, the optional features set out above in respect of the third aspect of the invention also apply equally well to the fourteenth aspect of the invention, except where clearly technically incompatible or where context dictates otherwise.

A fifteenth aspect of the present invention may provide a synthetic longitudinal data generation system comprising the TTS-GAN generation module of the twelfth aspect of the invention and the synthetic longitudinal data generation module of the thirteenth aspect of the invention. A sixteenth aspect of the invention may provide a survival analysis system comprising the synthetic longitudinal data generation module of the thirteenth aspect of the invention and the survival analysis module of the fourteenth aspect of the invention. The survival analysis system may further comprise the TTS-GAN generation module of the twelfth aspect of the invention.

Analogous to the seventh aspect of the present invention, a seventeenth aspect of the present invention may provide a data source configured to generate synthetic longitudinal data, the data source comprising: a receiver module configured to receive a request for synthetic longitudinal data; a synthetic context data generation module configured to apply a trained machine-learning model to input data comprising context data to generate synthetic context data having statistical properties corresponding to the statistical properties of the context data; a synthetic time-series data generation module configured to apply a trained transformer-based time-series generative adversarial network (TTS-GAN) generator to the generated synthetic context data to generate synthetic time-series data based on the synthetic context data; a transmitter module configured to output synthetic longitudinal data comprising the synthetic context data and the synthetic time-series data. The data source may further comprise an aggregation module configured to aggregate the synthetic context data and the synthetic time-series data to generate the synthetic longitudinal data. The data source may further comprise an interface module configured to receive the input data comprising the context data. The input data may be received from a data store in the data source.

Preferably, the data source may comprise a TTS-GAN generation module. The TTS-GAN generation module may be configured to train a transformer-based time-series generative adversarial network (TTS-GAN) to generate the trained TTS-GAN generator configured to generate the synthetic time-series data, the TTS-GAN comprising a TTS-GAN generator and a TTS-GAN discriminator, the TTS-GAN generation module comprising: an interface module configured to receive longitudinal training data comprising training context data and training time-series data; a training module configured to train the TTS-GAN using the longitudinal training data, the training module comprising a TTS-GAN generator optimizer and a TTS-GAN discriminator optimizer, wherein: (a) the TTS-GAN generator optimizer is configured to train the TTS-GAN generator to generate synthetic time-series data using the training context data and added noise; (b) the TTS-GAN discriminator optimizer is configured to train the TTS-GAN discriminator to discriminate between the training time-series data and the synthetic time-series data generated by the TTS-GAN generator; and (c) the training module is configured to alternate the operations executed in (a) and (b) until a predetermined end condition is met.

The data source may comprise a data source local server. The data source may further comprise one or more data source remote servers, for example a first data source remote server and a second data source remote server. The data source local server may comprise all of the modules of the data source. Alternatively, the data source local server may comprise a first sub-set of the modules, and the one or more data source remote servers may comprise a second sub-set of the modules. Preferably, the data source local server may comprise the receiver module, the synthetic context data generation module, the synthetic time-series data generation module, and the transmitter module, and the data source remote servers may comprise the TTS-GAN generation module.

It will be appreciated that the seventeenth aspect of the invention is a system which is adapted to execute the computer-implemented method of the seventh aspect of the invention. Accordingly, the optional features set out above in respect of the seventh aspect of the invention also apply equally well to the seventeenth aspect of the invention, except where clearly technically incompatible or where context dictates otherwise.

Analogous to an eighth aspect of the invention, an eighteenth aspect of the invention may provide a data consumer configured to receive synthetic longitudinal data, the data consumer comprising: a transmitter module configured to transmit a request for synthetic longitudinal data, and to transmit a transformer-based time-series generative adversarial network (TTS-GAN) comprising a TTS-GAN generator and a TTS-GAN discriminator; and a receiver module configured to receive synthetic longitudinal data comprising synthetic context data and synthetic time series data.

The data consumer may comprise a data consumer local server. The data consumer may further comprise a data consumer remote server. The data consumer local server may comprise the transmitter module, and the receiver module.

It will be appreciated that the eighteenth aspect of the invention is a system which is adapted to execute the computer-implemented method of the eighth aspect of the invention. Accordingly, the optional features set out above in respect of the eighth aspect of the invention also apply equally well to the eighteenth aspect of the invention, except where clearly technically incompatible or where context dictates otherwise.

Analogous to a ninth aspect of the invention, a nineteenth aspect of the invention provides a distributed system configured to request, generate, transmit and receive synthetic longitudinal data, comprising a data source and a data consumer. The data source may be a data source according to the seventeenth aspect of the present invention, and the data consumer may comprise a data consumer according to the eighteenth aspect of the invention.

It will be appreciated that the eighteenth aspect of the invention is a system which is adapted to execute the computer-implemented method of the ninth aspect of the invention. Accordingly, the optional features set out above in respect of the ninth aspect of the invention also apply equally well to the nineteenth aspect of the invention, except where clearly technically incompatible or where context dictates otherwise.

The invention includes the combination of the aspects and preferred features described except where such a combination is clearly impermissible or expressly avoided.

### BRIEF DESCRIPTION OF THE DRAWINGS

Embodiments of the present invention will now be described with reference to the accompanying drawings, in which:

FIG. 1 is a schematic drawing of a system which covers various aspects of the present invention.

FIG. 2A is a schematic drawing of a TTS-GAN generation module.

FIG. 2B is a schematic drawing of a synthetic longitudinal data generation module.

FIG. 2C is a schematic drawing of a survival analysis module.

FIG. 3 is a diagram illustrating the data flow during generation of a TTS-GAN, and synthetic longitudinal data.

FIG. 4 is flowchart illustrating a process by which a TTS-GAN is generated and trained.

FIG. 5 is a flowchart illustrating a process by which synthetic longitudinal data is generated using a TTS-GAN generator.

FIG. 6 is an example of a TTS-GAN.

FIG. 7 is a flowchart illustrating a survival analysis process.

FIG. 8 is an example of a probability vs. time curve.

FIG. 9 is an example of a distributed system comprising a data source and a data consumer.

### DETAILED DESCRIPTION OF THE DRAWINGS

Aspects and embodiments of the present invention will now be discussed with reference to the accompanying figures. Further aspects and embodiments will be apparent to those skilled in the art. All documents mentioned in this text are incorporated herein by reference.

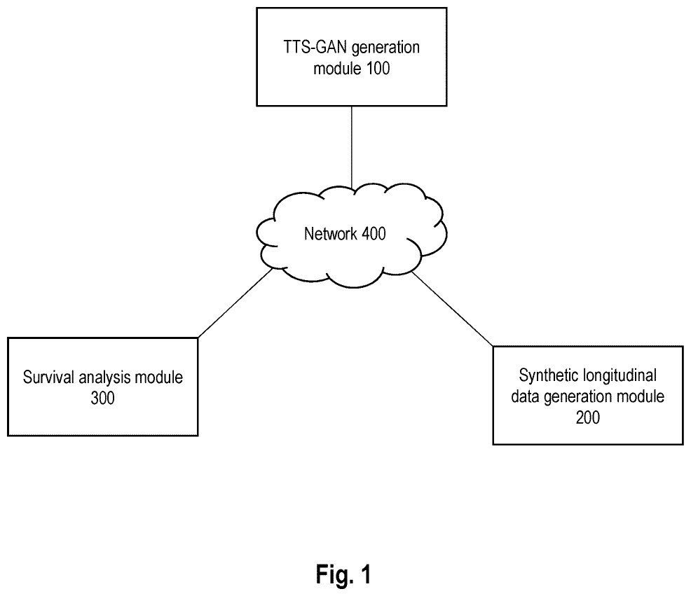

FIG. 1 shows an example of a system 10 which may be used to execute a computer-implemented method according to the present invention. The system 10 includes a TTS-GAN generation module 100, a synthetic longitudinal data generation module 200, and a survival analysis module 300. These modules may be implemented in various ways. For example, each could represent a separate piece of hardware, which may itself comprise a processor configured to execute the associated functions of those modules, and optionally a memory to store the results of the various processes performed. Alternatively, the module may be implemented in software, e.g. as different pieces of code, or scripts, each of which, when executed by a processor of an appropriate computer, is configured to cause that processor to execute the desired function. In some cases, some of the modules may be hardware modules, and others may be software modules. In some cases, the TTS-GAN generation module 100 and the synthetic longitudinal data generation module 200 may be located remotely from the survival analysis module 300. In this way, the computationally-demanding function of the TTS-GAN generation module 100 and the synthetic longitudinal data generation module 200 may be executed at e.g. a remote server or a cloud computing server, whereas the relatively less computationally-demanding survival analysis may be performed on e.g. a client device such as a laptop, smartphone, tablet, desktop or other suitable computing device. In FIG. 1, the TTS-GAN generation module 100, synthetic longitudinal data generation module 200, and survival analysis module 300 are shown connected by a network 400. This is not intended to limit the scope of this application to an arrangement in which all three components 100, 200, 300 are connected via a single network 400. For example, each pair of components may be connected by a separate network. Or, two components (e.g. the TTS-GAN generation module 100 and the synthetic longitudinal data generation unit 200) may be located on the same device, or network, and connect to the other component (e.g. the survival analysis module 300) via a network 400. The network 400 (or any other networks employed in the system 10) may be a wired network, such as a local-area network, or a wireless network such as the Internet, a Wi-Fi network, or a cellular network. The skilled person is well-aware of suitable variations.

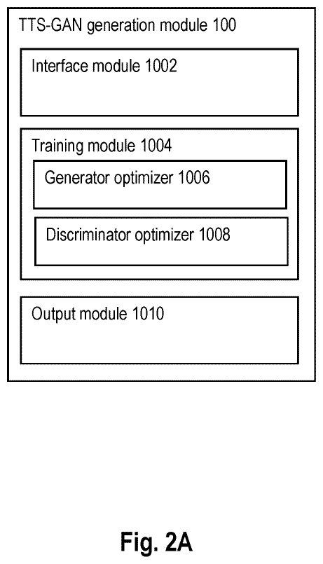

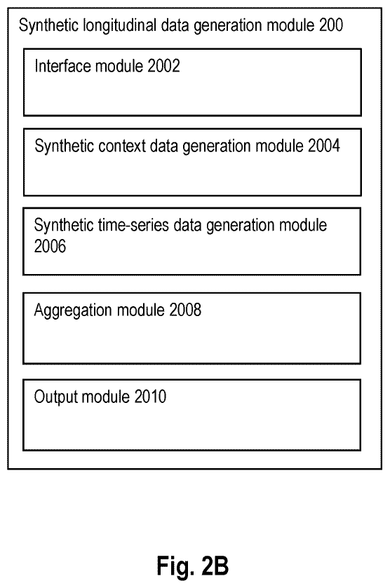

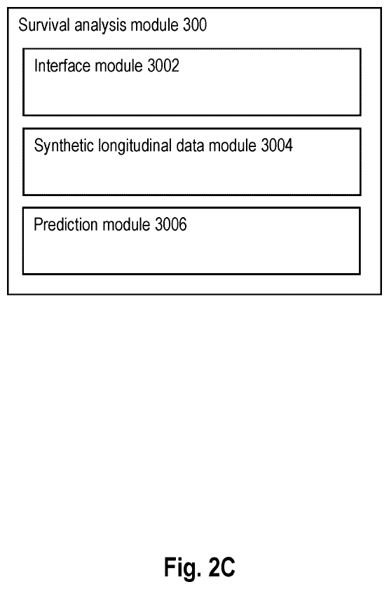

FIGS. 2A to 2C illustrate, respectively, the structure of the TTS-GAN generation module 100, the synthetic longitudinal data generation module 200, and the survival analysis module 300, described in turn below, before describing in detail the functions of each of these modules.

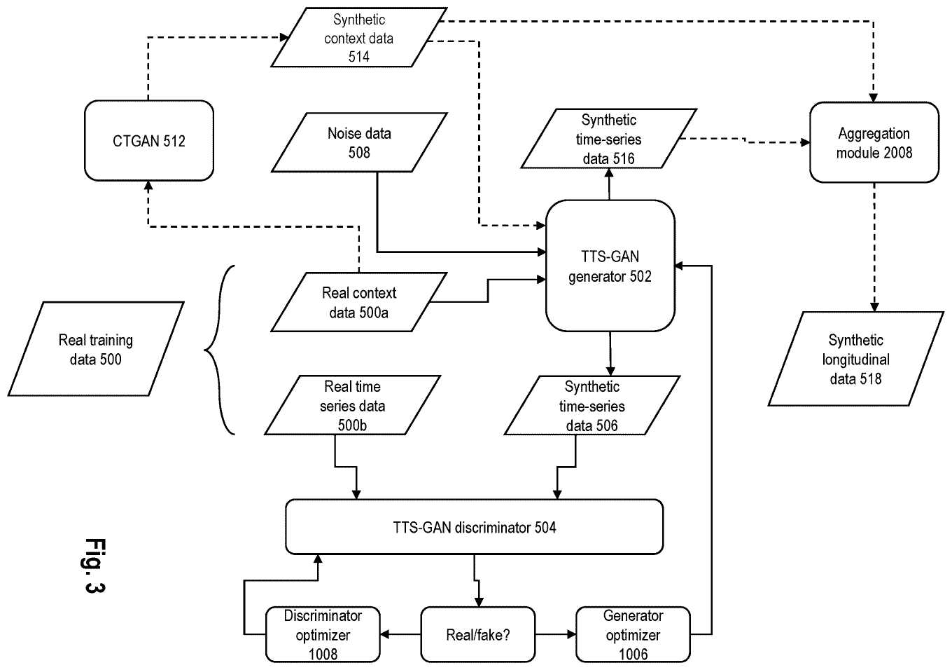

As shown in FIG. 2A, the TTS-GAN generation module 100 comprises an interface module 1002, a training module 1004 (comprising a TTS-GAN generator optimizer 1006 and a TTS-GAN discriminator optimizer 1008), and an output module 1010. The overall purpose of the TTS-GAN module 100 is to generate a TTS-GAN configured to generate synthetic longitudinal data, the TTS-GAN comprising a TTS-GAN generator and a TTS-GAN discriminator. As shown in FIG. 2B, the synthetic longitudinal data generation module 200 comprises an interface module 2002, a synthetic context data generation module 2004, a synthetic time-series data generation module 2006, an aggregation module 2008, and an output module 2010. The overall purpose of the synthetic longitudinal data generation module 200 is to generate synthetic longitudinal data for use in survival analysis. As shown in FIG. 3, the survival analysis module 300 comprises an interface module 3002, a synthetic longitudinal data module 3004, and a prediction module 3006. The overall purpose of the survival analysis module 300 is to predict a time at which a predetermined event will occur, or an amount of time remaining until the predetermined event will occur.

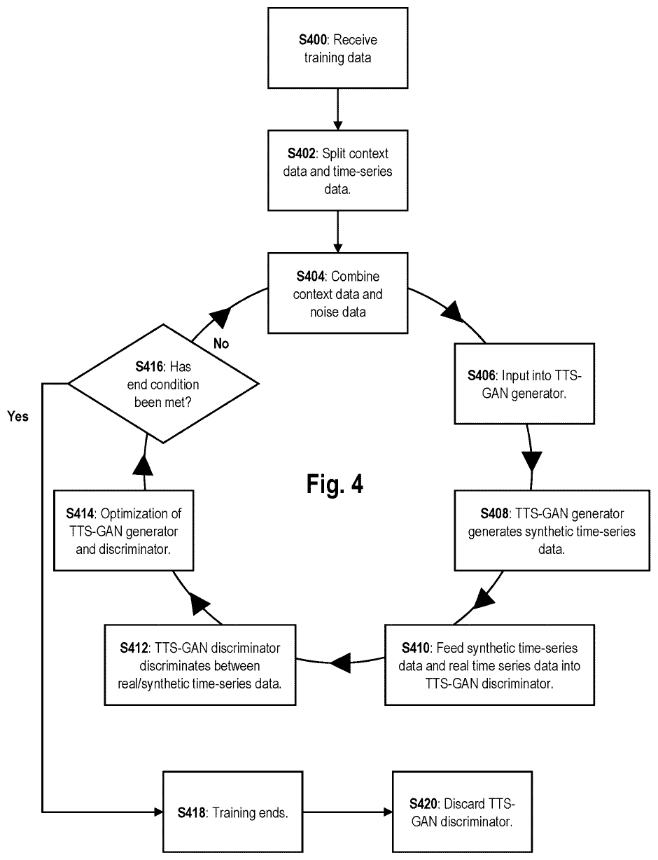

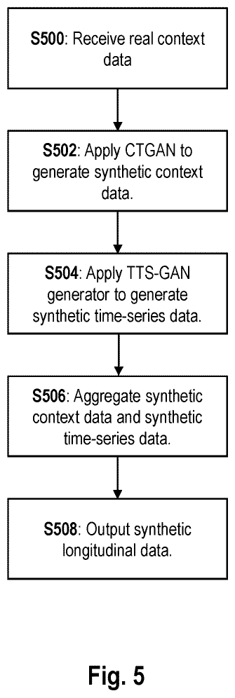

We now discuss the operation of the TTS-GAN generation module 100 and the synthetic longitudinal data generation module 200. FIG. 3 shows an overall schematic illustrating the data flow during the combined operation of these two modules 100, 200 in generating synthetic longitudinal data from a small amount of input training data. The solid lines denote the data flow arising from the functions performed by the TTS-GAN generation module 100 and the dotted lines denote the data flow arising from the functions performed by the synthetic longitudinal data generation module 200. The two functions can be considered separately. The data flow is described with reference to the flowcharts of FIGS. 4 and 5, which show respectively the series of steps performed by the TTS-GAN generation module 100 and the synthetic longitudinal data generation module 200.

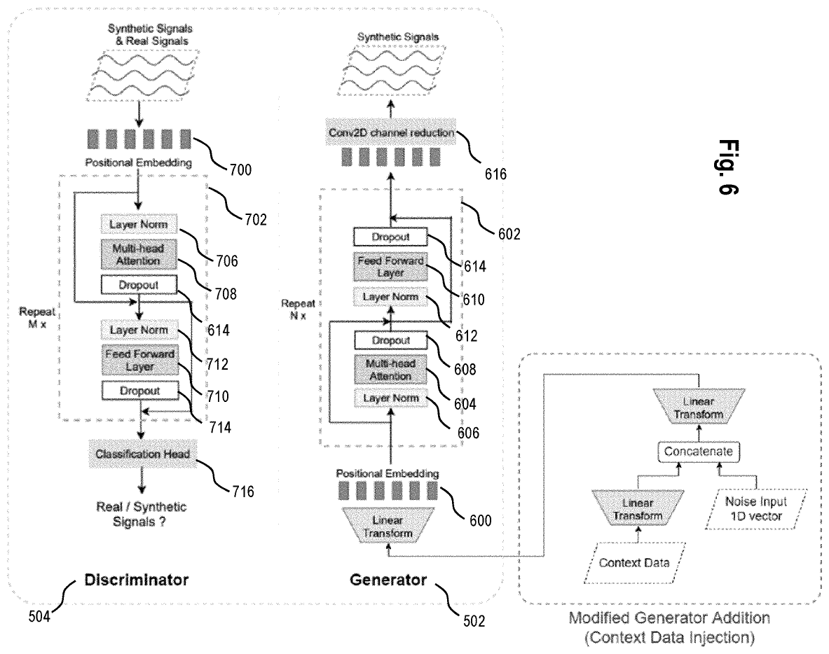

We begin with FIG. 4, and the operation of the TTS-GAN generation module 100. In step S400, training data (i.e. real data 500) is received at the interface module 1002 of the TTS-GAN generation module 100. The real data 500 comprises two elements: real context data 500a and real time-series data 500b. In step S402, the TTS-GAN generation unit splits the real context data 500a from the real time-series data 500b. At this point, broadly speaking, training of the TTS-GAN begins. Training of the TTS-GAN is adversarial, with the TTS-GAN generator 502 and TTS-GAN discriminator 504 being trained simultaneously. The TTS-GAN generator 502 is trained to generate synthetic time-series data 506 which is more realistic, and the TTS-GAN discriminator 504 trained to discriminate the ever more realistic synthetic time-series data 506 from the real time-series data 500b. This continuous process is executed by the training module 1004 of the TTS-GAN generation module 100 represented by the cycle in FIG. 4:
In step S404, the real context data 500a is combined with noise data 508. This may be done as shown in FIG. 6: a linear transform may be applied to the real context data 500a, the linear transform enabling the real context data 500a to be concatenated with the noise data 508. After concatenation, a further linear transform may be applied to the concatenation to generate input data. The input data may then be fed to the TTS-GAN generator 502 in step S406.
Then, in step S408, the TTS-GAN generator 502 may generate, based on the input data, synthetic time-series data 506. During the early stages of training, it must be appreciated that the synthetic time-series data 506 is likely to be very low quality, because the TTS-GAN generator 502 has not been trained to generate synthetic time-series data 506 based on non-time-series data. However, the quality of the generated synthetic time-series data 506 increases over time. The TTS-GAN generator 502 may generate a plurality of batches of synthetic time-series data 506 in step S408.
In step S410, the synthetic time-series data 506 and the real time-series data 500b are fed into the TTS-GAN discriminator 504. Then, in step S412, the TTS-GAN discriminator 504 attempts to discriminate 510 between the received synthetic time-series data 506 and the real time-series data 500b.
In step S414, based on the results of the attempts in step S412, and the corresponding generator loss and discriminator loss, the discriminator optimizer 1008 and the generator optimizer 1006 modify the weights used in the TTS-GAN generator 502 and the TTS-GAN discriminator 504 in order to improve their respective performances. There are six possible loss functions which might be used, including WGAN-GP.
In step S416, it is determined whether a predetermined end condition has been met (e.g. the training process has been executed for a threshold number of epochs such as no fewer than 2500 or no fewer than 3000, or if an equilibrium condition is reached). If so, the process ends in step S418, as it is assumed that the TTS-GAN generator 502 and the TTS-GAN discriminator 504 are performing optimally, in view of the training data received. If not, the process returns to step S404, and the cycle repeats using the updated weights of the TTS-GAN generator 502 and TTS-GAN discriminator 504.
In an optional step S420, after training is complete, the TTS-GAN discriminator 504 may be discarded, since it is not required for synthetic longitudinal data generation.

Before discussing the operation of the synthetic longitudinal data generation module 200 in detail with reference to FIG. 5, we discuss the structure of the TTS-GAN generator 502 and TTS-GAN discriminator 504 in more detail with reference to FIG. 6. The TTS-GAN generator 502 is shown on the right-hand side of the drawing. The structure of the input, described above with reference to S404 and S406 of FIG. 4 is shown in the dotted box labelled “Modified Generator Addition (Context Data Injection)”. After being input into the TTS-GAN generator 502, the data is fed through positional embedding layer 600. Then, the data is fed through compound generator unit 602. The compound generator unit 602 comprises a multi-head attention block 604 surrounded by a respective layer normalization layer 606 and dropout layer 608. After passing out of the dropout layer, the data is passed to feed-forward layer 610, also surrounded by a respective layer normalization layer 612 and dropout layer 614. The output of the compound generator unit 602 is then fed back into another compound generator unit 602—and this process is repeated N times. The output from the final compound generator unit 602 is then input into Conv2D channel reduction layer 616, which reduces the dimensionality of the synthetic data. This is then output as the synthetic time-series data 506.

The TTS-GAN discriminator 504 architecture is very similar to the architecture of the TTS-GAN generator 502, and is shown on the left-hand side of FIG. 6. The purpose of the TTS-GAN discriminator 504 is to discriminate (or to attempt to discriminate) between real time-series data 500b, and the generated synthetic time-series data 506. Accordingly, the input to the TTS-GAN discriminator 504 is a combination of real time-series data 500b signals and synthetic time-series data 506 signals. As with the TTS-GAN generator 502, the data is first fed through a positional embedding layer 700. Thereafter, the data is fed through compound discriminator unit 702. The compound discriminator unit 702 comprises a multi-head attention block 704 surrounded by a respective layer normalization layer 706 and dropout layer 708. After passing out of the dropout layer, the data is passed to feed-forward layer 710, also surrounded by a respective layer normalization layer 712 and dropout layer 14. The output of the compound discriminator unit 702 is then fed back into another compound discriminator unit 702—and this process is repeated M times. The output from the final compound discriminator unit 702 is then input into a classification head 716 which is configured to generate an output indicative of whether the input data was real time-series data 500b, or synthetic time-series data 506.

We now return to FIGS. 3 and 5, and discuss the operation of the synthetic longitudinal data generation module 200. In step S500, real context data 500a is received at the interface module 2002 of the synthetic longitudinal data generation module 200. In step S502, the synthetic context data generation module 2004 applies a machine-learning model, in the case of FIG. 3, CTGAN 512 to the real context data 500a to generate synthetic (“fake”) context data 514 which has statistical properties corresponding to the statistical properties of the received (“real”) context data 500a. Then, in step S504, the TTS-GAN generator 502, now trained, is applied by the synthetic time-series data generation module 2006 to the synthetic context data 514 in order to generate synthetic time-series data 516 based on the synthetic context data 514. Thereafter, in step S506, the synthetic context data 514 is aggregated with the synthetic time-series data 516 by the aggregation module 2008, to generate synthetic longitudinal data 518. The output module 2010 then outputs the synthetic longitudinal data 518, in step S508.

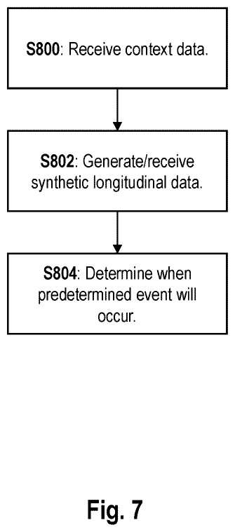

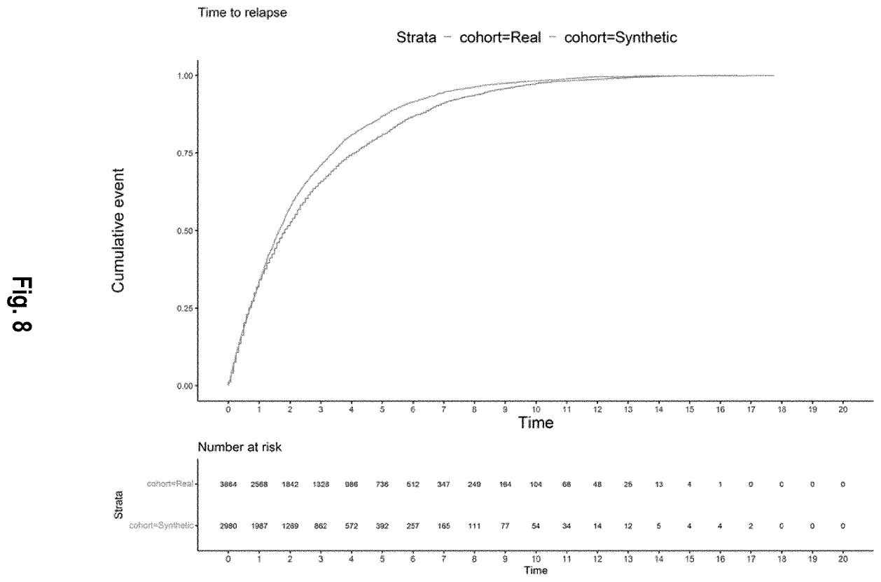

We now discuss the application of the synthetic longitudinal data 518 to survival analysis. FIG. 7 shows an example of a process which may be employed for executing survival analysis. In step S800, interface module 3002 of the survival analysis module 300 receives context data defining the values of a plurality of attributes for an entity. Then, in step S802, the synthetic longitudinal data module 3004 may either generate synthetic longitudinal data based on the received context data (e.g. by using the synthetic longitudinal data generation module 200), or the synthetic longitudinal data module may transmit the received context data to the synthetic longitudinal data generation module 200 and receive corresponding synthetic longitudinal data. The received synthetic longitudinal data is preferably representative of the change in the value of one or more predetermined variables. Finally, the prediction module 3006 may determine, based on the synthetic longitudinal data, the point in time at which a predetermined event will occur, or the amount of time remaining until the predetermined event will occur. The prediction module 3004 may do so by generating a plot of probability of the event occurring against time, such as in FIG. 8. Then, the point at which the probability exceeds a predetermined threshold value, it may be determined that that is the time at which the predetermined event takes place. The plot in FIG. 8 also illustrates that similar results are achieved using real data and synthetic data, demonstrating the efficacy of the present invention.

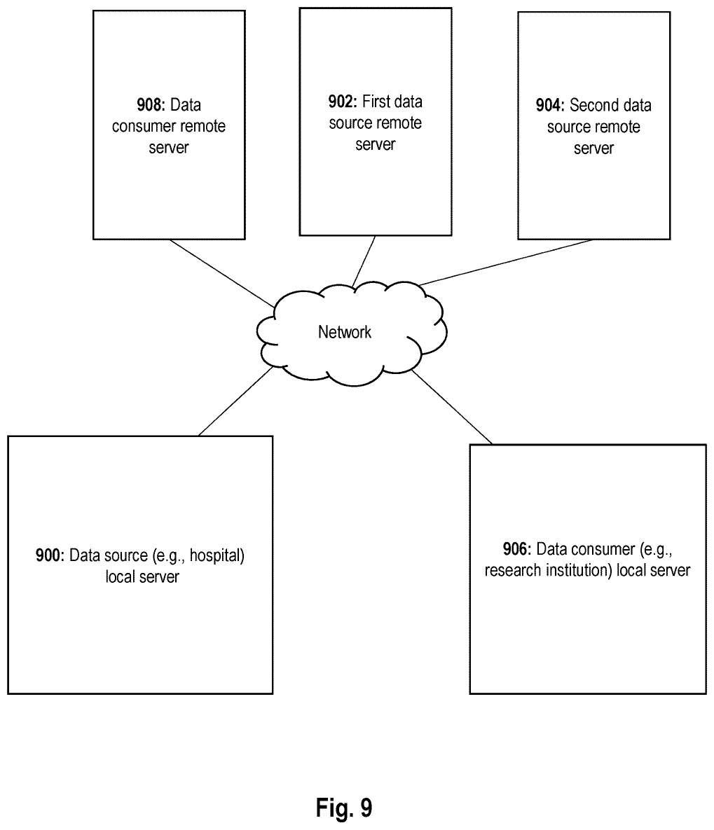

FIG. 9 shows an example of a distributed system comprising a data source and a data consumer. As can be seen in FIG. 9, the data source comprises a data source local server 900, a first data source remote server 902, and a second data source remote server 904. The data consumer comprises a data consumer local server 906 and a data consumer remote server 908. The servers are connected via a network, such as the internet.

The distributed system is configured to request, generate, transmit and receive synthetic longitudinal data.

We will now discuss the functionalities of the servers shown in the FIG. 9 embodiment. Nonetheless, it will be appreciated that the number of remote servers, and the functionalities of the servers, may vary between different embodiments.

In the example shown in FIG. 9, the data consumer local server 906 is configured to transmit a request for synthetic longitudinal data to the data source local server 900, to transmit an untrained TTS-GAN and an untrained machine-learning model to a data consumer remote server 908, and to receive synthetic longitudinal data from a second data source remote server 904. The data consumer remote server 908 is configured to receive the untrained TTS-GAN and the untrained machine-learning model from the data consumer local server 906, to store the untrained TTS-GAN and the untrained machine-learning model, and to transmit the untrained TTS-GAN and the untrained machine-learning model to the data source local server 900.

The data source local server 900 is configured to receive the request for synthetic longitudinal data from the data consumer, to receive the untrained TTS-GAN and untrained machine-learning model from the data consumer remote server 908, to transmit the untrained TTS-GAN and untrained machine learning model to the first data source remote server 902, to receive a trained TTS-GAN generator and trained machine-learning model from the first data source remote server 902, to apply the trained machine-learning model to input data comprising context data to generate synthetic context data having statistical properties corresponding to the statistical properties of the context data, to apply the trained TTS-GAN generator to the generated synthetic context data to generate synthetic time-series data based on the synthetic context data, and to transmit synthetic longitudinal data comprising the synthetic context data and the synthetic time-series data to the second data source remote server 904.

The first data source remote server 902 is configured to receive the untrained TTS-GAN and the untrained machine-learning model from the data source local server 900, to train the TTS-GAN and the untrained machine-learning model, and to transmit a trained TTS-GAN generator and the trained machine-learning model to the data source local server 900.

The second data source remote server 904 is configured to receive synthetic longitudinal data from the data source local server 900, to store the synthetic longitudinal data, and to transmit the synthetic longitudinal data to the data consumer local server 906.

We will now describe various functionalities of the servers in more detail.

The data consumer remote server 908 is configured to transmit the untrained TTS-GAN and the untrained machine-learning model to the data source local server 900 only when access to the data source remote server is provided to the data consumer local server 906. The data consumer remote server 908 is configured to provide access to the data source remote server when a key is received from the data source local server 900 which matches a predetermined data source key.

The first data source remote server 902 is configured to train the TTS-GAN by using longitudinal training data comprising training context data and training time-series data by: (a) training the TTS-GAN generator to generate synthetic time-series data using the training context data and added noise; (b) training the TTS-GAN discriminator to discriminate between the training time-series data and the synthetic time-series data generated by the TTS-GAN generator; (c) repeating steps (a) and (b) alternately until a predetermined end condition is met. In this way, a trained TTS-GAN generator configured to generate synthetic time-series data based on input data comprising context data is generated by the first data source remote server 902.

The second data source remote server 904 is configured to transmit the synthetic longitudinal data to the data consumer local server 906 only when access to the second data source remote server 904 is provided to the data consumer local server 906. The second data source remote server 904 is configured to provide access to the second data source remote server 904 when a key is received from the data consumer local server 906 which matches a predetermined data consumer key.

Accordingly, the distributed system shown in FIG. 9 may execute the follow method.

First, the data consumer transmits an untrained TTS-GAN and an untrained machine-learning model to the data consumer remote server 908. Later, the data consumer transmits request for synthetic data to the data source. Upon receiving the request for synthetic data, the data source may access the data consumer remote server 908 by inputting a predetermined data source key, and may download the untrained TTS-GAN and untrained machine-learning model from the data consumer remote server 908. Then, the data source local server 900 transmits the untrained models to the first data source remote server 902, where they are trained, and the trained TTS-GAN generator and trained machine-learning model are transmitted to the data source local server 900. At the data source local server 900, the trained machine-learning model is applied to input data comprising context data to generate synthetic context data having statistical properties corresponding to the statistical properties of the context data, and the trained TTS-GAN generator is applied to the generated synthetic context data to generate synthetic time-series data based on the synthetic context data. The data source local server 900 then transmits synthetic longitudinal data, comprising the synthetic context data and the synthetic time-series data, to the second data source remote server 904, where it is stored. The data consumer local server 906 may then access the second data source remote server 904 by inputting a predetermined data consumer key, and may download the synthetic longitudinal data.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A computer-implemented method of generating a transformer-based time-series generative adversarial network (TTS-GAN) configured to generate longitudinal synthetic data for use in survival analysis, a clinical trial, or clinical research, the TTS-GAN comprising a TTS-GAN generator and a TTS-GAN discriminator, the computer-implemented method comprising:
receiving longitudinal training data comprising context data and time-series data;
training the TTS-GAN using the longitudinal training data, wherein training the TTS-GAN comprises:
(a) training the TTS-GAN generator to generate synthetic time-series data using the context data and added noise;
(b) training the TTS-GAN discriminator to discriminate between the time-series data and the synthetic time-series data generated by the TTS-GAN generator;
(c) repeating steps (a) and (b) alternately until a predetermined end condition is met.

2. The computer-implemented method of claim 1, wherein:
training the TTS-GAN generator comprises:
receiving the context data and the noise data representing the noise;
applying a linear transform to the context data to generate linearly transformed context data, the linearly transformed context data concatenable with the noise data; and
concatenating the linearly transformed context data and the received noise data, to generation a concatenation.

3. The computer-implemented method of claim 2, further comprising:
applying a linear transform to the concatenation to generate a linearly transformed concatenation, wherein the linearly transformed concatenation forms the training input to the TTS-GAN generator.

4. The computer-implemented method of claim 1, wherein:
after training the TTS-GAN, the computer-implemented method further comprises discarding the TTS-GAN discriminator to retain only the TTS-GAN generator.

5. A computer-implemented method of generating synthetic longitudinal data for use in survival analysis, a clinical trial, or clinical research, using a generator of a transformer-based time-series generative adversarial network, the computer-implemented method comprising:
receiving input data, the input data comprising context data;
applying a machine-learning model to the context data to generate synthetic context data having statistical properties corresponding to the statistical properties of the received context data;
applying a trained TTS-GAN generator to the generated synthetic context data, the TTS-GAN generator configured to generate synthetic time-series data based on the synthetic context data, wherein the trained TTS-GAN generator is the generator of a TTS-GAN that has been trained according to the computer-implemented method of claim 1.

6. The computer-implemented method of claim 5, further comprising:
applying a linear transform to the context data to generate linearly transformed context data, the linearly transformed context data concatenable with the noise data; and
concatenating the linearly transformed context data and the received noise data, to generation a concatenation, the concatenation forming the input to the TTS-GAN generator.

7. The computer-implemented method of claim 6, further comprising:
applying a linear transform to the concatenation to generate a linearly transformed concatenation, wherein the linearly transformed concatenation forms the input to the TTS-GAN generator.

8. The computer-implemented method of claim 5, wherein:
the context data of the input data is the same as the context data of the training data.

9. The computer-implemented method of claim 5, further comprising:
aggregating the synthetic context data and the synthetic time-series data to generate the synthetic longitudinal data.

10. The computer-implemented method of claim 5, wherein:
the machine-learning model is a conditional generative adversarial network (CTGAN).

11. The computer-implemented method of claim 10, wherein:
the CTGAN has been trained according to the following computer-implemented method of generating synthetic context data:
receiving training context data in tabular form, the training data comprising column labels each defining a respective attribute of an entity, and records defining the values of the attributes for each of a respective plurality of entities;
training the GAN using the training context data, thereby generating a trained GAN, the trained GAN configured to generate synthetic context data having statistical properties corresponding to the statistical properties of the training context data.

12. The computer-implemented method of claim 10, wherein:
the CTGAN comprises:
a generator comprising one or more residual layers; and one or more linear layers; and
a discriminator comprising one or more linear layers; one or more rectified linear unit (ReLu) layers; and one or more dropout layers; and
training the GAN comprises:
(a) training the generator to minimize a generator loss function; and
(b) training the discriminator to minimize a discriminator loss function,
wherein steps (a) and (b) are repeated alternately until a predetermined end condition is met.

13. A computer-implemented method according to claim 5 wherein the computer-implemented method is a method of selecting a drug for further development, wherein a portion of the synthetic context data includes data indicating the provision of a given drug, and wherein the computer-implemented method further comprises;
determining whether a portion of the synthetic time-series data, the portion of the synthetic time-series data being generated from the portion of the synthetic context data, meets a pre-determined criterion; and,
in response to a determination that the portion of the synthetic time-series data meets the pre-determined criterion, selecting the given drug for further analysis and/or development.

14. A computer-implemented method of executing survival analysis to predict a time at which a predetermined event will occur, or an amount of time remaining until the predetermined event will occur, the computer-implemented method comprising:
receiving context data defining the values of a plurality of attributes for an entity;
generating, according to the computer-implemented method of claim 5, synthetic longitudinal data representative of the change in value of one or more predetermined variables;
and determining, based on the generated synthetic longitudinal data, the point in time at which the predetermined event will occur, or the amount of time remaining until the predetermined event will occur.

15. The computer-implemented method of claim 14, wherein:
determining the point in time or the amount of time remaining may comprise determining the point in time at which the probability of the predetermined event occurring is greater than or equal to a predetermined threshold.

16. The computer-implemented method of claim 14, wherein:
the predetermined event is one or more of: death, onset of a disease, onset of a symptom, relapse of a condition, metastasis, organ failure, or failure of a component.

17. A computer-implemented method according to claim 14, wherein the computer-implemented method is a method of selecting a drug for further development, wherein the predetermined event is a clinical event, wherein the point in time at which the predetermined event will occur, or the amount of time remaining until the predetermined event will occur corresponds to clinical survival analysis data, wherein a portion of the synthetic longitudinal data includes data indicating the provision of a given drug; and wherein the computer-implemented method further comprises:
determining whether a portion of the clinical survival analysis data, the portion of the clinical survival analysis data being generated from the portion of the synthetic longitudinal data, meets a pre-determined criterion; and,
in response to a determination that the clinical survival analysis data meets the pre-determined criterion, selecting the given drug for further analysis and/or development.

18. A computer-implemented method of generating synthetic longitudinal data, the computer-implemented method comprising:
receiving a request for synthetic longitudinal data;
applying a trained machine-learning model to input data comprising context data to generate synthetic context data having statistical properties corresponding to the statistical properties of the context data;
applying a trained transformer-based time-series generative adversarial network (TTS-GAN) generator to the generated synthetic context data to generate synthetic time-series data based on the synthetic context data; and,
outputting synthetic longitudinal data comprising the synthetic context data and the synthetic time-series data.

19. A computer-implemented method according to claim 18 further comprising calculating a statistical metric of the synthetic longitudinal data based on a comparison of the synthetic longitudinal data and real longitudinal data of which the context data was used to generate the synthetic longitudinal data.

20. A computer-implemented method according to claim 19 wherein the statistical metric includes a privacy metric and/or a quality metric.

21. A computer-implemented method according to claim 20, wherein the statistical metric includes a quality metric, and wherein calculating the quality metric comprises one or more of:
(a) conducting a univariate comparison;
(b) conducting a comparison test;
(c) conducting a machine-learning performance comparison; and,
(d) determining a distinguishability of the data sets.

22. The computer-implemented method according to claim 19, wherein the computer-implemented method further comprises:
a determination step of determining whether the statistical metric meets a predetermined threshold; and,
in response to determining that the statistical metric does not meet a predetermined threshold, an adjustment step of adjusting a value of a training parameter of the TTS-GAN and re-training the TTS-GAN using the adjusted training parameter value.

23. The computer-implemented method according to claim 22, wherein the determination step and the adjustment step are performed iteratively until it is determined that the statistical metric meets the predetermined threshold.

24. A computer-implemented method of receiving synthetic longitudinal data, the computer-implemented method comprising:
transmitting a request for synthetic longitudinal data;
transmitting a transformer-based time-series generative adversarial network (TTS-GAN) comprising a TTS-GAN generator and a TTS-GAN discriminator, the TTS-GAN configured to be trained using longitudinal training data comprising training context data and training time-series data by:
(a) training the TTS-GAN generator to generate synthetic time-series data using the training context data and added noise;
(b) training the TTS-GAN discriminator to discriminate between the training time-series data and the synthetic time-series data generated by the TTS-GAN generator;
(c) repeating steps (a) and (b) alternately until a predetermined end condition is met;
to generate a trained TTS-GAN generator configured to generate synthetic time-series data based on input data comprising context data; and,
receiving or accessing synthetic longitudinal data comprising synthetic context data and synthetic time series data, wherein the synthetic context data has been generated by applying a machine-learning model to input data comprising context data to generate synthetic context data having statistical properties corresponding to the statistical properties of the context data, and wherein the synthetic time-series data has been generated by the trained TTS-GAN generator.

25. A TTS-GAN generation module configured to generate a transformer-based time-series generative adversarial network (TTS-GAN) configured to generate longitudinal synthetic data for use in survival analysis, the TTS-GAN comprising a TTS-GAN generator and a TTS-GAN discriminator, the TTS-GAN generation module comprising:
an interface module configured to receive longitudinal training data comprising context data and time-series data;
a training module configured to train the TTS-GAN using the longitudinal training data, the training module comprising a TTS-GAN generator optimizer and a TTS-GAN discriminator optimizer,
wherein:
(a) the TTS-GAN generator optimizer is configured to train the TTS-GAN generator to generate synthetic time-series data using the context data and added noise;
(b) the TTS-GAN discriminator optimizer is configured to train the TTS-GAN discriminator to discriminate between the time-series data and the synthetic time-series data generated by the TTS-GAN generator; and
(c) the training module is configured to alternate the operations executed in (a) and (b) until a predetermined end condition is met.

26. A synthetic longitudinal data generation module configured to generate synthetic longitudinal data for use in survival analysis, the synthetic longitudinal data generation module comprising:
an interface module configured to receive input data, the input data comprising context data;
a synthetic context data generation module configured to apply a machine-learning model to the context data to generate synthetic context data having statistical properties corresponding to the statistical properties of the received context data;
a synthetic time-series data generation module configured to apply a trained TTS-GAN generator to the generated synthetic context data to generate synthetic time-series data based on the synthetic context data, wherein the TTS-GAN generator is the generator of a TTS-GAN generated by the TTS-GAN generation module of claim 25.

27. A survival analysis module configured to predict a time at which a predetermined event will occur, or an amount of time remaining until the predetermined event will occur, the survival analysis module comprising:
an interface module configured to receive context data defining the values of a plurality of attributes for an entity;
the synthetic longitudinal data generation module of claim 26, configured to generate synthetic longitudinal data representative of the change in value of one or more predetermined variables; and
a prediction module configured to determine, based on the generated synthetic longitudinal data, the point in time at which the predetermined event will occur, or the amount of time remaining until the predetermined event will occur.
